(19) United States
(12) Patent Application Publication
(10) Pub. No.: US 2022/0193415 A1
(43) Pub. Date: Jun. 23, 2022

(54) CLOSED LOOP CONTROL SYSTEM

(71) Applicant: UNIVERSITY OF LOUISVILLE RESEARCH FOUNDATION, INC., LOUISVILLE, KY (US)

(72) Inventors: SUSAN J. HARKEMA, Louisville, KY (US); ENRICO REJC, Louisville, KY (US); CLAUDIA ANGELI, Louisville, KY (US); CHARLES H. HUBSCHER, Louisville, KY (US); APRIL N. HERRITY, Peewee Valley, KY (US); YANGSHENG CHEN, Louisville, KY (US); SEVDA G. ASLAN, Louisville, KY (US)

(73) Assignee: UNIVERSITY OF LOUISVILLE RESEARCH FOUNDATION, INC., LOUISVILLE, KY (US)

(21) Appl. No.: 17/601,186

(22) PCT Filed: Apr. 4, 2020
(86) PCT No.: PCT/US2020/026761

§ 371 Date: Oct. 4, 2021

Related U.S. Application Data
(60) Provisional application No. 62/945,702, filed on Dec. 9, 2019.
(60) Provisional application No. 62/829,901, filed on Apr. 5, 2019.

(51) Int. Cl. A61N 1/36; A61N 1/05

(52) U.S. Cl. CPC A61N 1/36062; A61N 1/36007; A61N 1/0551; A61N 1/36117; A61N 1/36139

(57) ABSTRACT

A closed loop system for control of spinal cord epidural stimulation includes a second controller hosting software for receiving, from at least one sensor, physiological data from a subject, generating a stimulation configuration based on the data, and transmitting the configuration to a first controller which operatively causes a neurostimulator to apply the stimulation configuration to the subject, the physiological results of such stimulation are monitored by the at least one sensor.

This application claims the benefit of U.S. provisional patent application Ser. No. 62/945,702 filed 9 Dec. 2019 for CLOSED LOOP CONTROL SYSTEM and U.S. provisional patent application Ser. No. 62/829,901 filed 5 Apr. 2019 for CLOSED LOOP CONTROL SYSTEM, both of which are incorporated herein by reference.

### FIELD OF THE INVENTION

A closed loop system for control of spinal cord epidural stimulation includes a second controller hosting software for receiving, from at least one sensor, physiological data from a subject, generating a stimulation configuration based on the data, and transmitting the configuration to a first controller which operatively causes a neurostimulator to apply the stimulation configuration to the subject, the physiological results of such stimulation are monitored by the at least one sensor.

### BACKGROUND OF THE INVENTION

Individuals with motor complete spinal cord injury (SCI) are unable to stand, walk, or move their lower limbs voluntarily; this condition drastically affects their quality of life and implies severe limitations for functional recovery. Bladder dysfunction post-injury manifests as an inability to efficiently store and empty the bladder and continually ranks as a top problematic issue impacting overall quality of life. Compromised cardiovascular function is also a critical consequence of SCI and is interrelated with urinary tract impairment. Bladder complications often trigger cardiovascular events such as autonomic dysreflexia, resulting in dramatic elevations in blood pressure. Targeted spinal cord epidural stimulation (scES) aimed at improving cardiovascular and bladder function in individuals with severe SCI is being investigated. Through detailed mapping experiments, it has been shown that the appropriate selection of stimulation configurations (amplitude, pulse width, frequency, and anode/cathode assignment) is critical for generating effective tasks in both cardiovascular and bladder function. However, the lack of closed-looped control of neuromodulation for the heart and bladder currently limit its therapeutic relevance. Further, given the dynamic regulatory interplay between bladder and cardiovascular systems, an ability to generate complex multi-system stimulation patterns in real-time is needed for improving regulation and function. For example, in individuals with SCI or other neurological disorder resulting in impaired lower urinary tract (LUT) function, rising bladder pressure can dangerously increase blood pressure, limiting the effectiveness of scES activation triggered from blood pressure signals alone. For these reasons, integrated use of scES to improve cardiovascular and bladder function in individuals with SCI or other neurological disorder remains limited in the home and in the community.

### SUMMARY

By using scES devices that are already FDA-approved (for use in chronic pain in non-SCI individuals), and independent development environment (IDE) approved for SCI or other neurological disorders resulting in impaired LUT function, the inventors improve existing technology by upgrading the programming and wireless communication platforms associated with a neurostimulator and make them specifically suitable for use by individuals with impaired LUT function. Coupling and integrating existing technology to monitor and interact with the stimulator can generate a seamless system capable of delivering multiple training paradigms across multiple physiological systems. The current gap that places the burden on the user to adjust and monitor stimulation and physiological parameters remains one of the most important limiting factors in the effective utilization of scES technology outside of the laboratory. This development will interact with spinal cord stimulation systems, both currently known and those developed hence, to facilitate the implementation of training paradigms for the recovery of motor and autonomic function in individuals with neurological disorders, such as SCI, and promote safe long-term use of the technology in the home and community. To this end, the disclosed invention provides a flexible communication platform specific for individuals with neurological disorder resulting in impaired LUT function, allowing for the evaluation of integrated technology in the individuals and allowing for the longitudinal evaluation of therapeutic benefits of scES in individuals over time.

It will be appreciated that the various systems and methods described in this summary section, as well as elsewhere in this application, can be expressed as a large number of different combinations and sub-combinations. All such useful, novel, and inventive combinations and sub-combinations are contemplated herein, it being recognized that the explicit expression of each of these combinations is unnecessary.

### BRIEF DESCRIPTION OF THE DRAWINGS

A better understanding of the present invention will be had upon reference to the following description in conjunction with the accompanying drawings.

FIG. 1 depicts a schematic diagram of the closed loop control system for scES.

FIG. 2 is a flowchart illustrating an embodiment of a predictive learning algorithm for providing neurostimulation to control blood pressure.

FIG. 3 is a flowchart illustrating an embodiment of a predictive learning algorithm for providing neurostimulation to control bladder storage and voiding.

FIG. 4 is a flowchart illustrating an embodiment of a predictive learning algorithm for providing neurostimulation to control bladder storage.

### DETAILED DESCRIPTION OF THE PREFERRED EMBODIMENTS

For the purposes of promoting an understanding of the principles of the invention, reference will now be made to selected embodiments illustrated in the drawings and specific language will be used to describe the same. It will nevertheless be understood that no limitation of the scope of the invention is thereby intended; any alterations and further modifications of the described or illustrated embodiments, and any further applications of the principles of the invention as illustrated herein are contemplated as would normally occur to one skilled in the art to which the invention relates. At least one embodiment of the invention is shown in great detail, although it will be apparent to those skilled in the relevant art that some features or some combinations of features may not be shown for the sake of clarity.

Any reference to “invention” within this document is a reference to an embodiment of a family of inventions, with no single embodiment including features that are necessarily included in all embodiments, unless otherwise stated. Furthermore, although there may be references to “advantages” provided by some embodiments of the present invention, other embodiments may not include those same advantages, or may include different advantages. Any advantages described herein are not to be construed as limiting to any of the claims.

Specific quantities (spatial dimensions, dimensionless parameters, etc.) may be used explicitly or implicitly herein, such specific quantities are presented as examples only and are approximate values unless otherwise indicated. Discussions pertaining to specific compositions of matter, if present, are presented as examples only and do not limit the applicability of other compositions of matter, especially other compositions of matter with similar properties, unless otherwise indicated. Unless stated otherwise, explicit approximate quantities (e.g., about 1; approximately 20) refer to a range of ±5% of the recited quantities (e.g., “about 1” refers to 0.95 to 1.05; “approximately 20” refers to the range of 19 to 21).

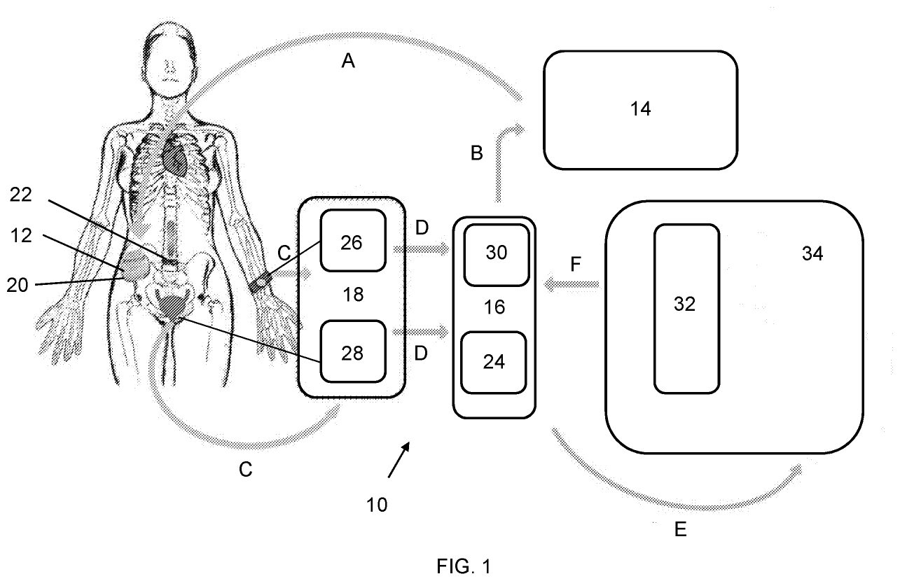

Referring to FIG. 1, an intersystem closed-loop control system 10 is configured to modulate bladder, cardiovascular, and autonomic systems in a subject having an implanted neurostimulator. In some embodiments, the closed-loop control system 10 includes a neurostimulator 12, a first controller 14, a second controller 16, and at least one sensor 18. A typical neurostimulator 12 includes an electric pulse generator 20 connected to leads or an electrode array 22 for delivering scES. The first controller 14 is in electronic communication with and operatively controls the implanted neurostimulator 12, as indicated by line A. In one exemplary embodiment, the implanted neurostimulator 12 is a Medtronic™ Intellis™ neurostimulator and the first controller 14 is a neurostimulator controller, such as a Medtronic™ Intellis™ clinician programmer or MyStim™ programmer. In other embodiments, other neurostimulators and associated programmers may be used, or similar devices, now known or developed hence, which serve a similar function. The second controller 16 is, in some embodiments, a mobile device, such as a smartphone, tablet computer, or other portable computing device including a processor, a non-transitory computer readable storage medium, and means for electronic communication to and from the second controller. The second controller 16 hosts operative software 24, referred to herein as the Participant Interface Control System (PICS) software application. The PICS software 24 is in electronic communication, preferably wirelessly, with the first controller 14, as indicated by line B. The PICS software 24 communicates with the first controller 14, delivering instructions to adjust the stimulation parameters of scES provided by the implanted neurostimulator. In an exemplary embodiment, the first controller 14 implements a TCP server, while the second controller 16, via the PICS software 24, implements a TCP client which sends stimulation parameters and other instructions to the first controller 14 via a wifi network or other wireless connection. The PICS software 24 is also in electronic communication, preferably wirelessly, with the at least one sensor 18 for monitoring physiological data of the subject. In some embodiments, the at least one sensor 18 includes a first sensor 26 and a second sensor 28. In the embodiment depicted in FIG. 1, the first sensor 26 is a wearable blood pressure (BP) monitor, and the second sensor 28 is a wireless bladder pressure monitor. In other embodiments, the first sensor 26 and second sensor 28 may be different devices for monitoring physiological data from the patient (lines C), preferably real-time continuous physiological data, and the control system 10 may include third, fourth, or additional sensors. Preferably, each sensor monitors a different physiological characteristic (e.g., BP, bladder pressure, bladder capacity, etc.).

The PICS software 24 receives physiological data regarding the subject in electronic form from the at least one sensor 18 (e.g., blood pressure monitor 24, bladder pressure monitor 26, or others, as shown by lines D), then applies a predictive learning algorithm 30 to identify optimal stimulation configurations for neuromodulation of bladder and cardiovascular function, then communicates the stimulation configurations to the first controller 14 to apply the stimulation configurations to the neurostimulator. The predictive learning algorithm 30 may be software hosted on the second controller 16 as in the embodiment shown in FIG. 1, or hosted on a separate computing system in communication with the second controller 16. In some embodiments, the closed-loop control system 10 includes a plurality of predictive learning algorithms 28, each governing the delivery of scES for a different purpose (e.g., controlling blood pressure, bladder control, bladder voiding, specific motor muscle activities such as standing, sitting, walking, etc.). The control system 10 thus comprises a closed loop, where sensors 18 monitor the subject's physiological data (lines C), the second controller 16 receives the data (lines D), the PICS software 24 hosted on the second controller 16 generates scES stimulation configurations based at least in part on the physiological data, the scES stimulation configurations are transmitted to the first controller 14 (line B), which delivers the scES stimulation configurations to the implanted neurostimulator 12 (line A), which applies the scES stimulation configurations to the subject, the results of which are reflected in the patient's physiological data, which are monitored by the sensors 18 (lines C), and the loop continues. Particularly in embodiments where the predictive learning algorithm 30 is hosted on the second controller 16, and wherein the second controller 16 is a mobile computing device, the entire closed loop control system (i.e., neurostimulator, sensor, first controller, and second controller) 10 may be portable such that the subject may benefit from automatically determined and applied scES to modulate bladder, cardiovascular, and autonomic system function without being tied to a fixed location.

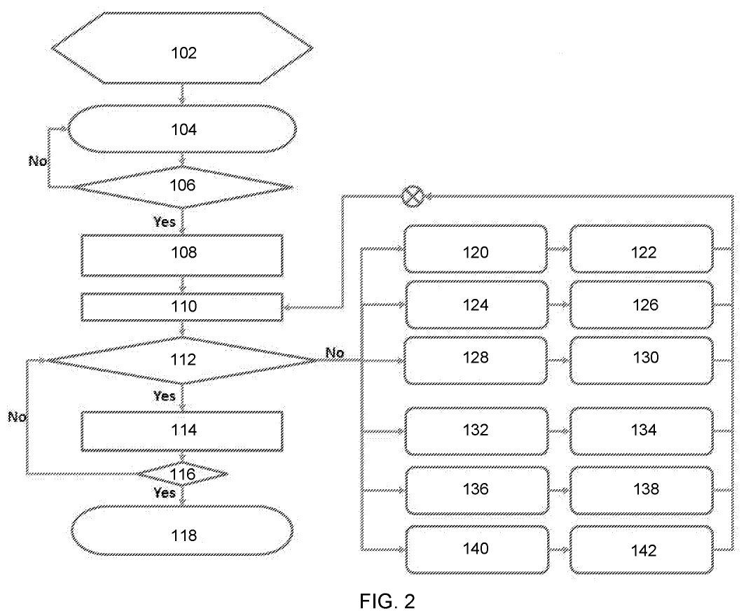

Exemplary predictive learning algorithms 28 for control of blood pressure, control of bladder voiding, and control of bladder storage are illustrated in FIGS. 2, 3 and 4, respectively. With respect to these algorithms, BP=blood pressure, HR=heart rate, SBP=systolic blood pressure, AD=autonomic dysreflexia, and detrusor refers to the detrusor muscle (smooth muscle found in the wall of the bladder which relaxes to allow the bladder to expand to store urine, then contracts to allow urine to flow into the urethra). The predictive learning algorithms are created by extracting neural signal features and patterns of specific bladder and cardiovascular responses from a multi-system mapping database. In the instant algorithms, first and second scES configurations, referred to as StimConfigP1 and StimConfigP2, are determined through BP and bladder mapping, respectively. For example, in the algorithm for controlling BP shown in FIG. 2, StimConfig 1 is a set of scES parameters (e.g., amplitude, pulse width, frequency, anode/cathode assignment, etc.) for the implanted neurostimulator to regulate the subject's blood pressure. StimConfig2 is a set of scES parameters to reduce muscle activity, namely, to suppress the muscle contractions caused by the stimulation of StimConfig1. The muscle contractions are monitored by electromyogram (EMG)

Referring now to FIG. 2, an exemplary first embodiment 100 of predictive learning algorithm 30 is configured to provide neurostimulation to control blood pressure. Conceptually, this algorithm directs the delivery of scES to maintain the subject's SBP within a predetermined range, e.g., between 110 mmHg and 120 mmHg. This first embodiment 100 includes the steps of:

102—Identifying StimConfig1 to regulate BP; Identifying StimConfig2 to regulate muscle activity using mapping process (described below); proceed to step 104.

104—Initiate BP and HR recording; proceed to step 106. BP and heart rate may be monitored substantially continuously or at preset intervals (e.g., five readings every three minutes, or other time interval, as appropriate).

106—Determine if most recent SBP reading is <110 mmHg; if no, return to step 104; if yes, proceed to step 108.

108—Initiate neurostimulation by gradually increasing stimulation amplitude to an initial amplitude over a predetermined duration (e.g., increase stimulation amplitude from 0 mA to 5 mA over a one minute duration) for StimConfigP1 and StimConfigP2, then proceed to step 110.

110—Wait two minutes from completing the previous step, then measure BP (in other embodiments, different time intervals may be used), then proceed to step 112.

112—Determine if SBP reading in step 110 is >=110 mmHg and <=120 mmHg; if yes, proceed to step 114; if no, proceed to decision tree in steps 120, 124, 128, 132, 136, and 140.

114—BP is currently within the predetermined range; measure BP again five minutes after enacting step 112 (in other embodiments, different time intervals may be used), then proceed to step 116.

116—Determine if SBP reading in step 114 is >=110 mmHg and <=120 mmHg; if yes, proceed to step 118; if no, return to step 112.

118—End neurostimulation by decreasing stimulation amplitude to 0.0 mA for both StimConfig1 and StimConfig2.

120—If SBP>130 mmHg, proceed to step 122.

122—Decrease stimulation amplitude for StimConfigP1 and StimConfigP2 by 0.9 mA (or to 0.0 mA, if current amplitude is less than 0.9 mA), then proceed to step 110.

124—If SBP>125 mmHg and SBP<=130 mmHg, proceed to step 126.

126—Decrease stimulation amplitude for StimConfigP1 and StimConfigP2 by 0.6 mA (or to 0.0 mA, if current amplitude is less than 0.6 mA), then proceed to step 110.

128—If SBP>120 mmHg and SBP<=125 mmHg, proceed to step 130.

130—Decrease stimulation amplitude for StimConfigP1 and StimConfigP2 by 0.3 mA (or to 0.0 mA, if current amplitude is less than 0.3 mA), then proceed to step 110.

132—If SBP>=105 mmHg and SBP<110 mmHg, proceed to step 134.

134—Increase stimulation amplitude for StimConfigP1 and StimConfigP2 by 0.6 mA, then proceed to step 110.

136—If SBP>=100 mmHg and SBP<105 mmHg, proceed to step 138.

138—Increase stimulation amplitude for StimConfigP1 and StimConfigP2 by 0.9 mA, then proceed to step 110.

140—If SBP<100 mmHg, proceed to step 142.

142—Increase stimulation amplitude for StimConfigP1 and StimConfigP2 by 1.2 mA, then proceed to step 110.

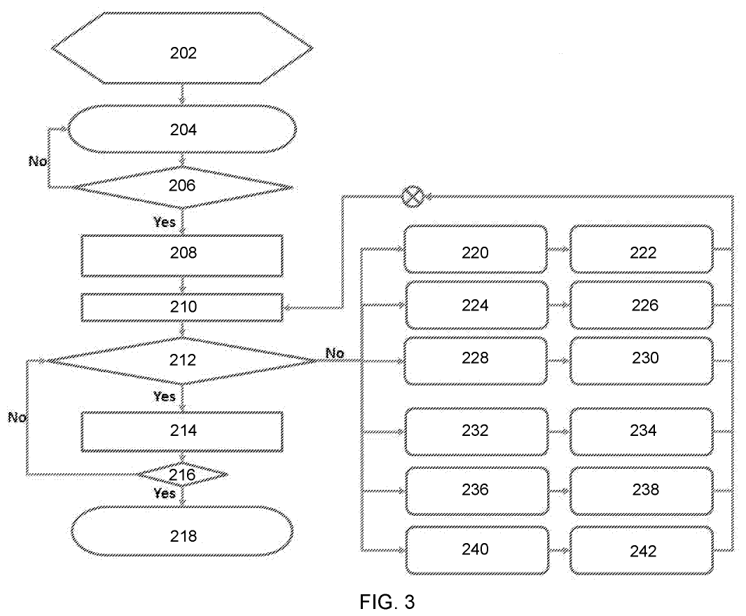

Referring now to FIG. 3, an exemplary second embodiment 200 of predictive learning algorithm 30 is configured to provide neurostimulation to void a subject's bowels. Conceptually, this algorithm directs the delivery of scES to cause a subject's bladder to void by increasing detrusor voiding pressure to a predetermined threshold, e.g., >40 cmH₂O, while maintaining the subject's SBP within a predetermined range, e.g., between 110 mmHg and 120 mmHg. This second embodiment 200 includes the steps of:

202—Identifying StimConfig1 to regulate bladder voiding; Identifying StimConfig2 to regulate cardiovascular responses during voiding using mapping process (described below); proceed to step 204. In some embodiments, the regulated cardiovascular responses are BP and heart rate.

204—Initiate monitoring of detrusor voiding pressure and CV status; proceed to step 206. In some embodiments, detrusor voiding pressure in monitored substantially continuously via catheter during filling cystometry. Volume infused into the bladder during cystometry provides an estimation of capacity, while total capacity is captured at the end of the filling cycle. CV status, e.g., BP and heart rate, may be monitored substantially continuously or at preset intervals.

206—Determine if most recent SBP reading is <110 mmHg, if bladder capacity is 400 ml-450 ml, and if detrusor voiding pressure is <40 cmH₂O; if no, return to step 204; if yes, proceed to step 208.

208—Initiate neurostimulation by gradually increasing stimulation amplitude to an initial amplitude over a predetermined duration (e.g., increase stimulation amplitude from 0 mA to 5 mA over a one minute duration) for StimConfigP1 and StimConfigP2, then proceed to step 210.

210—Monitor detrusor voiding pressure and continue to measure BP; then proceed to step 212. The time intervals between monitoring depend upon the capacity of the subject's bladder (e.g., more rapid monitoring for subjects with lower capacity).

212—Determine if SBP reading in step 210 is >=110 mmHg and <=120 mmHg, and if detrusor voiding pressure is <40 cmH₂O; if yes, proceed to step 214; if no, proceed to decision tree in steps 220, 224, 228, 232, 236, and 240. Note that steps 220, 224, 228, 232, and 240 apply different criteria to SBP and detrusor voiding pressure, such that one step may be enacted with respect to modulation of StimConfig1 and a different step may be enacted with respect to StimConfig2.

214—BP is within the predetermined range and detrusor voiding pressure is less than the threshold value; measure BP again five minutes after enacting step 212 (in other embodiments, different time intervals may be used), then proceed to step 216.

216—Determine if SBP reading in step 214 is >=110 mmHg and <=120 mmHg; if yes, proceed to step 218; if no, return to step 212.

218—End neurostimulation by decreasing stimulation amplitude to 0.0 mA for both StimConfig1 and StimConfig2.

220—If SBP>130 mmHg or if detrusor voiding pressure>40 cmH₂O, proceed to step 222.

222—Decrease stimulation amplitude for StimConfigP1 by 0.1 mA to 0.9 mA if detrusor voiding pressure>40 cmH₂O (or to 0.0 mA, if current amplitude is less than the amount of decrease), and decrease stimulation amplitude for StimConfigP2 by 0.1 mA to 0.9 mA (or to 0.0 mA, if current amplitude is less than the amount of decrease) if SBP>130 mmHg, then proceed to step 210.

224—If SBP>125 mmHg and SBP<=130 mmHg, proceed to step 226.

226—Decrease stimulation amplitude for StimConfigP2 by 0.1 mA to 0.6 mA (or to 0.0 mA, if current amplitude is less than 0.6 mA), then proceed to step 210.

228—If SBP>120 mmHg and SBP<=125 mmHg, proceed to step 230.

230—Decrease stimulation amplitude for StimConfigP2 by 0.1 to 0.3 mA (or to 0.0 mA, if current amplitude is less than 0.3 mA), then proceed to step 210.

232—If SBP>=105 mmHg and SBP<110 mmHg, proceed to step 234.

234—Increase stimulation amplitude for StimConfigP2 by 0.6 mA, then proceed to step 210.

236—If SBP>=100 mmHg and SBP<105 mmHg, or if detrusor voiding pressure>20 cmH₂O and detrusor voiding pressure<40 cmH₂O, proceed to step 238.

238—Increase stimulation amplitude for StimConfigP1 by 0.9 mA if detrusor pressure>20 cmH₂O and detrusor voiding pressure<40 cmH₂O, and increase stimulation amplitude for StimConfigP2 by 0.9 mA if SBP>=100 mmHg and SBP<105 mmHg, then proceed to step 210.

240—If SBP<100 mmHg or if detrusor voiding pressure<20 cmH₂O, proceed to step 242.

242—Increase stimulation amplitude for StimConfigP1 by 1.2 mA if detrusor pressure <20 cmH₂O, and increase stimulation amplitude for StimConfigP2 by 1.2 mA if SBP<100 mmHg, then proceed to step 210.

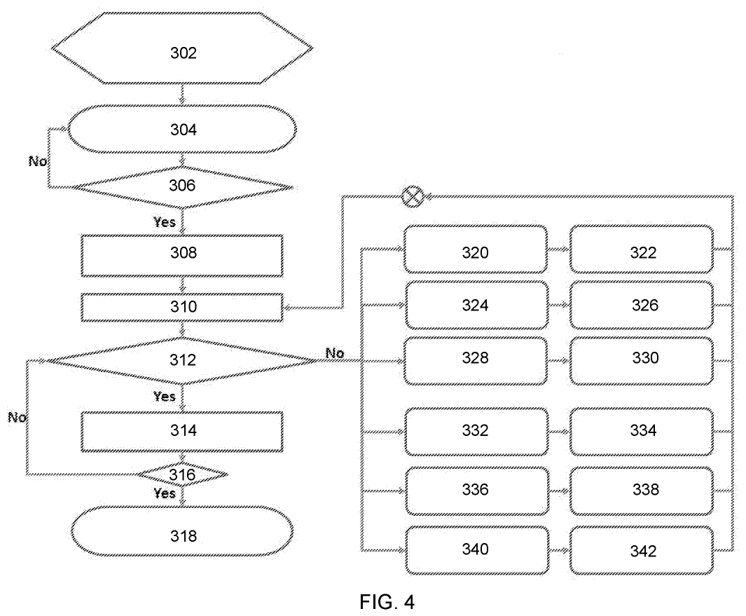

Referring now to FIG. 4, an exemplary third embodiment 300 of predictive learning algorithm 30 is configured to provide neurostimulation to regulate a subject's bladder storage. Conceptually, this algorithm directs the delivery of scES to allow a subject's bladder to fill, as indicated by a gradual increase in detrusor filling pressure, e.g., <10 cm H₂O and an increase in sphincter EMG, while maintaining the subject's SBP within a predetermined range, e.g., between 110 mmHg and 120 mmHg. However, if the subject's BP remains elevated, scES is terminated and the bladder is emptied (by catheter, if necessary). This third embodiment 300 includes the steps of:

302—Identifying StimConfig1 to regulate bladder storage; Identifying StimConfig2 to regulate cardiovascular responses during bladder distention using mapping process (described below); proceed to step 204. In some embodiments, the regulated cardiovascular responses are BP and heart rate.

304—Initiate monitoring of detrusor filling pressure and CV status; proceed to step 306. In some embodiments, detrusor filling pressure in monitored substantially continuously via catheter during filling cystometry. Volume infused into the bladder during cystometry provides an estimation of capacity, while total capacity is captured at the end of the filling cycle. For clarification, detrusor filling pressure refers to pressure in the bladder as it expands and relaxes with urine (or saline, in cystometry). Detrusor voiding pressure is pressure generated by contraction of the detrusor muscle during voiding. While the bladder is filling, detrusor filling pressure remains relatively low until the time of voiding. While the bladder is voiding, the onset of detrusor voiding pressure is relatively high and decreases as the bladder empties. CV status, e.g., BP and heart rate, may be monitored substantially continuously or at preset intervals.

306—Determine if most recent SBP reading is <110 mmHg, if bladder capacity is 400 ml-450 ml, and if detrusor filling pressure is <10 cmH₂O; if no, return to step 304; if yes, proceed to step 308.

308—Initiate neurostimulation by gradually increasing stimulation amplitude to an initial amplitude over a predetermined duration (e.g., increase stimulation amplitude from 0 mA to 5 mA over a one minute duration) for StimConfigP1 and StimConfigP2, then proceed to step 310.

310—Monitor detrusor filling pressure and continue to measure BP; then proceed to step 312. The time intervals between monitoring depend upon the capacity of the subject's bladder (e.g., more rapid monitoring for subjects with lower capacity).

312—Determine if SBP reading in step 310 is >=110 mmHg and <=120 mmHg, and if detrusor filling pressure is <10 cmH₂O; if yes, proceed to step 314; if no, proceed to decision tree in steps 320, 324, 328, 332, 336, and 340. Note that steps 320, 324, 328, 332, and 340 apply different criteria to SBP and detrusor filling pressure, such that one step may be enacted with respect to modulation of StimConfig1 and a different step may be enacted with respect to StimConfig2.

314—BP is within the predetermined range and detrusor filling pressure is beneath the threshold value; measure BP again five minutes after enacting step 312 (in other embodiments, different time intervals may be used), then proceed to step 316.

316—Determine if SBP reading in step 314 is >=110 mmHg and <=120 mmHg; if yes, proceed to step 318; if no, return to step 312.

318—End neurostimulation by decreasing stimulation amplitude to 0.0 mA for both StimConfig1 and StimConfig2.

320—If SBP>130 mmHg or if detrusor filling pressure>40 cmH₂O, proceed to step 322.

322—Decrease stimulation amplitude for StimConfigP1 by 0.1 mA to 0.9 mA if detrusor filling pressure>40 cmH₂O (or to 0.0 mA, if current amplitude is less than 0.9 mA), and decrease stimulation amplitude for StimConfigP2 by 0.1 mA to 0.9 mA (or to 0.0 mA, if current amplitude is less than 0.9 mA) if SBP>130 mmHg, then proceed to step 310.

324—If SBP>125 mmHg and SBP<=130 mmHg or if detrusor filling pressure>20 mmH₂O and detrusor filling pressure<=40 cm H₂O, proceed to step 326.

326—Decrease stimulation amplitude for StimConfigP1 by 0.1 mA to 0.6 mA if detrusor filling pressure>20 mmH₂O and detrusor pressure<=40 cm H₂O (or to 0.0 mA, if current amplitude is less than 0.6 mA), and decrease stimulation amplitude for StimConfigP2 by 0.1 mA to 0.6 mA (or to 0.0 mA, if current amplitude is less than 0.6 mA) if SBP>125 mmHg and SBP<=130 mmHg, then proceed to step 310.

328—If SBP>120 mmHg and SBP<=125 mmHg, or if detrusor filling pressure>10 mmH₂O and detrusor filling pressure<=20 cm H₂O, proceed to step 330.

330—Decrease stimulation amplitude for StimConfigP1 by 0.1 mA to 0.3 mA if detrusor filling pressure>10 mmH₂O and detrusor filling pressure<=20 cm H₂O (or to 0.0 mA, if current amplitude is less than 0.3 mA), and decrease stimulation amplitude for

StimConfigP2 by 0.1 mA to 0.3 mA (or to 0.0 mA, if current amplitude is less than 0.3 mA) if SBP>120 mmHg and SBP<=125 mmHg, then proceed to step 310.

332—If SBP>=105 mmHg and SBP<110 mmHg, proceed to step 334.

334—Increase stimulation amplitude for StimConfigP2 by 0.1 mA to 0.6 mA, then proceed to step 310.

336—If SBP>=100 mmHg and SBP<105 mmHg, proceed to step 338.

338—Increase stimulation amplitude for StimConfigP2 by 0.1 mA to 0.9 mA, then proceed to step 310.

340—If SBP<100 mmHg, proceed to step 342.

342—Increase stimulation amplitude for StimConfigP2 by 1.2 mA, then proceed to step 310.

During enactment of the exemplary predictive learning algorithms 100, 200, 300 the amplitudes of StimConfigP1 and StimConfigP2 are preferably kept as close as possible. If the amplitudes cannot be kept equal, the amplitude of StimConfigP2 should preferably be greater than the amplitude of StimConfigP1. In some embodiments, if BP is maintained in the predetermined range for at least ten minutes (or in other embodiments, at least 20 minutes or other designated time period), and step 106, 206, 306 indicates a SBP<110 mmHg, the system will wait two minutes and determine the SBP again to confirm that the value remains below the optimized range before proceeding to corresponding step 108, 208, 308. If symptoms of AD, leg spasm, or abdominal spasm occur (typically detected by changes in BP), the amplitude of StimConfigP1 and StimConfigP2 are decreased until the symptoms cease.

While the disclosed exemplary embodiments 100, 200, 300 contemplate a predetermined range of SBP between 110 mmHg and 120 mmHg, in other embodiments, the range may be between 100 mmHg and 110 mmHg, 105 mmHg and 115 mmHg, 115 mmHg and 125 mmHg, 120 mmHg and 130 mmHg, 100 mmHg and 120 mmHg, 105 mmHg and 125 mmHg, or 110 mmHg and 130 mmHg, as appropriate for the individual subject. While the disclosed exemplary embodiments 200 and 300 contemplate triggering various steps based on detrusor voiding pressures and detrusor filling pressures, respectively, of <10 cmH₂O, <20 cmH₂O, or <40 cmH₂O, in other embodiments, other pressure values may be used, such as, for example, <5 cmH₂O, <15 cmH₂O, <15 cmH₂O, <25 cmH₂O, <30 cmH₂O, <35 cmH₂O, <45 cmH₂O, or <50 cmH₂O, as appropriate for the individual subject. It should also be understood that the exemplary embodiments 100, 200, 300 are representative of predictive learning algorithms 30, and that similar algorithms may be used which modify frequency, pulse width, pulse duration, selection of electrode for activation, or other parameter relevant to scES based on one or more monitored physiological states, instead of or in addition to modifying amplitude. Also, while the exemplary embodiments 100, 200, 300 modify scES parameters based on monitored SBP, detrusor filling pressure, and detrusor voiding pressure, it should be understood that other physiological states (e.g, diastolic blood pressure, heart rate, body temperature, breathing rate, etc.) may be monitored and utilized in other embodiments of predictive learning algorithms 30.

Referring again to FIG. 1, some embodiments of the closed-loop control system 10 includes a multi-system mapping database 32. The mapping database 32 may be hosted on a separate computing system 34 in communication with the second controller 16, as shown in FIG. 1. In certain embodiments, second controllers 16 used by a plurality of subjects are each in communication with the same computing system 34 and receive and share data from the same multi-system mapping database 32. The mapping database 32 includes a plurality of programs governing scES for cardiovascular, bladder, or bowel control, or scES for eliciting complex muscle movements, such as standing, stepping, and others. The programs are generated based on data from multiple research and clinical studies. One program may involve multiple cohorts. For example, a program for controlling bladder capacity may include three cohorts: Bladder Capacity, CV Decrease, Bladder Pressure. Each cohort includes information such as scES parameters (e.g., configurations of amplitude, pulse width, frequency, anode/cathode assignment, durations, and other scES delivery characteristics), and physiology responses such as, for example, increased bladder capacity, decreased SBP, and lowered bladder pressure as correlated with scES delivery. In some embodiments, researchers and clinicians can search the database 32 with selected criteria, and develop an algorithm, such as the exemplary algorithms 100, 200, 300 disclosed herein. For example, researchers and clinicians may search the database 32 for a scES program to control BP with a predetermined range of 110 mmHg to 120 mmHg, then develop an algorithm to vary the application of scES parameters in the program based on the physiological data obtained by monitoring the subject (e.g., raise and lower scES amplitude based on the subject's SBP). In other embodiments, machine learning techniques may be used by the PICS software 24 to automatically search the database 32 for appropriate scES programs (line F on FIG. 1) and develop algorithms based on monitored physiological data.

The extracted neural signal features (e.g., BP, heart rate, detrusor voiding pressure, detrusor filling pressure) detected by the at least one sensor 18 are integrated with the multi-system mapping database 32 to optimize stimulation configurations that improve bladder and cardiovascular function. The PICS software 24 transmits to the multi-system mapping database 32 data regarding scES applied to the subject and physiological data before and after application of scES to increase the content of the database and allow refinement and improvement to scES programs based on participant responses to stimulation (line E on FIG. 1). PICS software 24 not only regulates multi-system function with closed-loop control through scES, but it also identifies the optimal stimulation parameters that can target the dynamic regulatory interplay between systems in order to improve functional outcomes (i.e. both bladder storage and emptying while controlling autonomic fluctuations in blood pressure). The PICS software 24 will adjust stimulation parameters to regulate the cardiovascular function and bladder function, as bladder function can affect cardiovascular function as well. For example, PICS software 24 used with a first subject, a 300 lb. male, may obtain an algorithm 30 to control BP, such as the first embodiment 100, from the database 32. The PICS software 24 may deliver the stimulation parameters to first controller 14, which causes the neurostimulator 12 to deliver the scES, as monitored by the at least one sensor 18. According to the first embodiment 100, the PICS software 24 will raise or lower the amplitude until it reaches stimulation parameters that maintains the subject's SBP within the predetermined range of 110 mmHg to 120 mmHg. Those stimulation parameters are then electronically delivered to the mapping database 32 together with relevant physiological data regarding the subject. When PICS software 24 is later used to control the BP of a second subject, another 300 lb. male, instead of selecting the first embodiment 100, which has initial StimConfigP1 and StimConfigP2 amplitudes of 5 mA, the PICS software 24 could select the algorithm developed for the first subject, and begin with StimConfigP1 and StimConfigP2 values optimized for the first subject, which presumably would be a better fit for the second subject.

While the exemplary embodiments 100, 200, 300 discuss varying the amplitude of scES stimulation programs, it should be understood that in other embodiments, the PICS software 24 may vary the pulse width, pulse duration, pulse frequency, or other scES parameter to achieve the desired result (e.g., maintain SBP within the predetermined range, increase or decrease detrusor filling or voiding pressure, etc.) In preferred embodiments, the PICS software 24 is configured to receive verbal commands from subjects, which may be helpful for individuals with SCI or other neurological disorder who have limited hand function.

The disclosed multi-system mapping database 32 may be embodied in computer program instructions stored on a non-transitory computer readable storage medium configured to be executed by the computing system 34. The disclosed PICS software may be embodied in computer program instructions stored on a non-transitory computer readable storage medium configured to be executed by the second controller 16. The computing system 34, as well as first controller 14 and second controller 16, will typically include a processor in communication with a memory, and a network interface. Power, ground, clock, and other signals and circuitry are not discussed, but will be generally understood and easily implemented by those ordinarily skilled in the art. The processor, in some embodiments, is at least one microcontroller or general purpose microprocessor that reads its program from memory. The memory, in some embodiments, includes one or more types such as solid-state memory, magnetic memory, optical memory, or other computer-readable, non-transient storage media. In certain embodiments, the memory includes instructions that, when executed by the processor, cause the computing system to perform a certain action. The computing system 34, first controller 14, and second controller 16 also preferably include a network interface connecting the computing system to a data network for electronic communication of data between the various devices attached to the network as indicated in FIG. 1. In certain embodiments, the processor includes one or more processors and the memory includes one or more memories. In some embodiments, computing system is defined by one or more physical computing devices as described above. In other embodiments, the computing system may be defined by a virtual system hosted on one or more physical computing devices as described above.

Various aspects of different embodiments of the present disclosure are expressed in paragraphs X1, X2, and X3 as follows:

X1: One embodiment of the present disclosure includes a control system for spinal cord epidural stimulation (scES), comprising: a neurostimulator configured to apply scES to a subject; at least one sensor for monitoring a physiological state of the subject; a first controller in electronic communication with the neurostimulator, the first controller being configured to control the scES applied by the neurostimulator; and a second controller in electronic communication with the at least one sensor and the first controller; wherein the second controller includes a processor and a non-transitory computer readable storage medium having computer program instructions stored thereon that, when executed by the processor, cause the processor to perform the following instructions: receiving, from the at least one sensor, data describing the physiological state; generating a scES configuration based at least in part on the received data; and transmitting the scES configuration to the first controller.

X2: Another embodiment of the present disclosure includes a method of improving bladder function in a subject with impaired bladder control, the method comprising: applying spinal cord epidural stimulation (scES) to the subject according to a first scES configuration; applying scES to the subject according to a second scES configuration; monitoring a first physiological state of the subject after applying scES according to the first scES configuration; monitoring a second physiological state of the subject after applying scES according to the second scES configuration; modifying at least one of the first scES configuration based at least in part on the first physiological state and the second scES configuration based at least in part on the second physiological state.

X3: A non-transitory computer readable storage medium having computer program instructions stored thereon that, when executed by a processor, cause the processor to perform the following instructions: receiving, from at least one sensor, data describing a physiological state of a subject; generating a spinal cord epidural stimulation (scES) configuration based at least in part on the received data; and transmitting the scES configuration to a neurostimulator controller.

Yet other embodiments include the features described in any of the previous paragraphs X1, X2, or X3 as combined with one or more of the following aspects:

Wherein the at least one sensor includes a first sensor for monitoring a first physiological state of the subject and a second sensor for monitoring a second physiological state of the subject, and wherein the first physiological state and second physiological state are not identical.

Wherein said receiving comprises receiving, from the first sensor, data describing the first physiological state and receiving, from the second sensor, data describing the second physiological states; and wherein said generating comprises generating a first scES configuration based at least in part of the received data describing the first physiological state and generating a second scES configuration based at least in part on the received data describing the second physiological state; and wherein transmitting the scES comprises transmitting the first scES configuration to the first controller and transmitting and the second scES to the first controller.

Wherein the computer program instructions, when executed by the processor, cause the processor to performed the following additional instructions: receiving, from the at least one sensor, data describing the physiological state after application of scES by the neurostimulator; and modifying the scES configuration based at least in part on the data describing the physiological state after application of scES by the neurostimulator.

Wherein the first physiological state and second physiological state are not identical.

Wherein applying scES according to the first scES configuration and applying scES according to the second scES configuration occur simultaneously.

Wherein applying scES according to the first scES configuration is applying at an intensity sufficient to enact one of the following: increase or decrease detrusor filling pressure, increase or decrease detrusor voiding pressure, increase or decrease blood pressure, and increase or decrease heart rate.

Wherein applying scES according to the second scES configuration is applying at an intensity sufficient to enact one of the following: increase or decrease detrusor filling pressure, increase or decrease detrusor voiding pressure, increase or decrease blood pressure, and increase or decrease heart rate.

Wherein one of the first physiological state and the second physiological state is selected from the group consisting of blood pressure, heart rate, detrusor filling pressure, and detrusor voiding pressure.

Wherein modifying the first scES configuration based at least in part on the first physiological state comprising modifying the first scES if the first physiological state is not within a predetermined range or if the first physiological state is above or below a threshold value.

Wherein modifying the second scES configuration based at least in part on the second physiological state comprising modifying the second scES if the second physiological state is not within a predetermined range or if the second physiological state is above or below a threshold value.

Wherein the receiving comprises receiving first sensor data from a first sensor, the first sensor data describing a first physiological state and receiving second sensor data from a second sensor, the second sensor data describing a second physiological state.

Wherein the first physiological state and second physiological state are not identical.

Wherein the generating a scES configuration comprises generating a first scES configuration based at least in part on the first sensor data and generating a second scES configuration based at least in part on the second sensor data.

Wherein the first scES configuration is modified based on the first sensor data.

Wherein the second scES configuration is modified based on the second sensor data.

Wherein the step of receiving data occurs both before and after the step of transmitting the scES configuration.

Wherein the step of generating the scES configuration is based at least in part on the received data being inside or outside a predetermined range.

Wherein the step of generating the scES configuration is based at least in part on the received data being above or below a threshold value.

Wherein the computer program instructions, when executed by the processor, cause the processor to performed the following additional instructions: receiving, from the at least one sensor, data describing the physiological state after application of scES; and modifying the scES configuration based at least in part on the data describing the physiological state after application of scES.

Wherein the computer program instructions, when executed by the processor, cause the processor to performed the following additional instructions: receiving, from the at least one sensor, data describing the physiological state after application of scES; and transmitting instructions to cease scES to the neurostimulator controller if the physiological state is maintained within a predetermined range.

The foregoing detailed description is given primarily for clearness of understanding and no unnecessary limitations are to be understood therefrom for modifications can be made by those skilled in the art upon reading this disclosure and may be made without departing from the spirit of the invention.

What is claimed is:

1) A control system for spinal cord epidural stimulation (scES), comprising:
a neurostimulator configured to apply scES to a subject;
at least one sensor for monitoring a physiological state of the subject;
a first controller in electronic communication with the neurostimulator, the first controller being configured to control the scES applied by the neurostimulator; and
a second controller in electronic communication with the at least one sensor and the first controller;
wherein the second controller includes a processor and a non-transitory computer readable storage medium having computer program instructions stored thereon that, when executed by the processor, cause the processor to perform the following instructions:
receiving, from the at least one sensor, data describing the physiological state;
generating a scES configuration based at least in part on the received data; and
transmitting the scES configuration to the first controller.

2) The control system of claim 1, wherein the at least one sensor includes a first sensor for monitoring a first physiological state of the subject and a second sensor for monitoring a second physiological state of the subject, and wherein the first physiological state and second physiological state are not identical.

3) The control system of claim 2,
wherein said receiving comprises receiving, from the first sensor, data describing the first physiological state and receiving, from the second sensor, data describing the second physiological states; and
wherein said generating comprises generating a first scES configuration based at least in part of the received data describing the first physiological state and generating a second scES configuration based at least in part on the received data describing the second physiological state; and
wherein transmitting the scES comprises transmitting the first scES configuration to the first controller and transmitting and the second scES to the first controller.

4) The control system of claim 1, wherein the computer program instructions, when executed by the processor, cause the processor to performed the following additional instructions:
receiving, from the at least one sensor, data describing the physiological state after application of scES by the neurostimulator; and
modifying the scES configuration based at least in part on the data describing the physiological state after application of scES by the neurostimulator.

5) A method of improving bladder function in a subject with impaired bladder control, the method comprising:
applying spinal cord epidural stimulation (scES) to the subject according to a first scES configuration;
applying scES to the subject according to a second scES configuration;
monitoring a first physiological state of the subject after applying scES according to the first scES configuration;
monitoring a second physiological state of the subject after applying scES according to the second scES configuration;
modifying at least one of
the first scES configuration based at least in part on the first physiological state and
the second scES configuration based at least in part on the second physiological state.

6) The method of claim 5, wherein the first physiological state and second physiological state are not identical.

7) The method of claim 5, wherein applying scES according to the first scES configuration and applying scES according to the second scES configuration occur simultaneously.

8) The method of claim 5, wherein applying scES according to the first scES configuration is applying at an intensity sufficient to enact one of the following: increase or decrease detrusor filling pressure, increase or decrease detrusor voiding pressure, increase or decrease blood pressure, and increase or decrease heart rate.

9) The method of claim 5, wherein applying scES according to the second scES configuration is applying at an intensity sufficient to enact one of the following: increase or decrease detrusor filling pressure, increase or decrease detrusor voiding pressure, increase or decrease blood pressure, and increase or decrease heart rate.

10) The method of claim 5, wherein one of the first physiological state and the second physiological state is selected from the group consisting of blood pressure, heart rate, detrusor filling pressure, and detrusor voiding pressure.

11) The method of claim 5, wherein modifying the first scES configuration based at least in part on the first physiological state comprising modifying the first scES if the first physiological state is not within a predetermined range or if the first physiological state is above or below a threshold value.

12) The method of claim 5, wherein modifying the second scES configuration based at least in part on the second physiological state comprising modifying the second scES if the second physiological state is not within a predetermined range or if the second physiological state is above or below a threshold value.

11) A non-transitory computer readable storage medium having computer program instructions stored thereon that, when executed by a processor, cause the processor to perform the following instructions:
receiving, from at least one sensor, data describing a physiological state of a subject;
generating a spinal cord epidural stimulation (scES) configuration based at least in part on the received data; and
transmitting the scES configuration to a neurostimulator controller.

12) The non-transitory computer readable storage medium having computer program instructions stored thereon of claim 11, wherein the receiving comprises receiving first sensor data from a first sensor, the first sensor data describing a first physiological state and receiving second sensor data from a second sensor, the second sensor data describing a second physiological state.

13) The non-transitory computer readable storage medium having computer program instructions stored thereon of claim 12, wherein the first physiological state and second physiological state are not identical.

14) The non-transitory computer readable storage medium having computer program instructions stored thereon of claim 12, wherein the generating a scES configuration comprises generating a first scES configuration based at least in part on the first sensor data and generating a second scES configuration based at least in part on the second sensor data.

15) The non-transitory computer readable storage medium having computer program instructions stored thereon of claim 12, wherein the first scES configuration is modified based on the first sensor data.

16) The non-transitory computer readable storage medium having computer program instructions stored thereon of claim 12, wherein the second scES configuration is modified based on the second sensor data.

16) The non-transitory computer readable storage medium having computer program instructions stored thereon of claim 11, wherein the step of receiving data occurs both before and after the step of transmitting the scES configuration.

17) The computer readable storage medium having computer program instructions stored thereon of claim 11, wherein the step of generating the scES configuration is based at least in part on the received data being inside or outside a predetermined range.

18) The computer readable storage medium having computer program instructions stored thereon of claim 11, wherein the step of generating the scES configuration is based at least in part on the received data being above or below a threshold value.

19) The non-transitory computer readable storage medium having computer program instructions stored thereon of claim 11, wherein the computer program instructions, when executed by the processor, cause the processor to performed the following additional instructions:
receiving, from the at least one sensor, data describing the physiological state after application of scES; and
modifying the scES configuration based at least in part on the data describing the physiological state after application of scES.

20) The non-transitory computer readable storage medium having computer program instructions stored thereon of claim 11, wherein the computer program instructions, when executed by the processor, cause the processor to performed the following additional instructions:
receiving, from the at least one sensor, data describing the physiological state after application of scES; and
transmitting instructions to cease scES to the neurostimulator controller if the physiological state is maintained within a predetermined range.
